(19) United States
(12) Patent Application Publication
(10) Pub. No.: US 2018/0353115 A1
(43) Pub. Date: Dec. 13, 2018

(54) PORTABLE DEVICE CASE FOR PULSE OXIMETRY MEASUREMENTS

(71) Applicant: KONINKLIJKE PHILIPS N.V., EINDHOVEN (NL)

(72) Inventors: John CRONIN, BONITA SPRINGS, FL (US); Michael Glynn D'ANDREA, BONITA SPRINGS, FL (US)

(21) Appl. No.: 15/777,332

(22) PCT Filed: Nov. 22, 2016
(86) PCT No.: PCT/EP2016/078362

§ 371 Date: May 18, 2018

Related U.S. Application Data
(60) Provisional application No. 62/259,060, filed on Nov. 24, 2015.

(30) Foreign Application Priority Data
Mar. 4, 2016 (EP) 16158771.2

(51) Int. Cl. A61B 5/1455; A61B 5/00

(52) U.S. Cl. CPC A61B 5/14551; A61B 5/746; A61B 5/0002; A61B 2560/045; A61B 5/742; A61B 2560/0431

(57) ABSTRACT

A method for using pulse oximetry connected to a portable device case comprising: acquiring a pulse oximetry data using the pulse oximeter; storing the acquired pulse oximetry data in a memory in the portable device case; connecting a patient monitoring device to the portable device case via a patient monitoring device port; and transmitting the stored pulse oximetry data to the patient monitoring device.

### BACKGROUND OF THE INVENTION

Pulse oximetry has become an important technique for monitoring patients and is now widely-used to assist in diagnosing various medical conditions. In addition, portable versions of pulse oximetry-based devices have further expanded the technique's applicability. Using portable pulse oximeters, patients can freely move and perform their regular activities. In this way, healthcare providers can monitor the patient outside a hospital setting.

The use of a portable monitoring device for interfacing with a mobile communication device such as a smartphone is well known in the art. Examples of portable monitoring devices are wrist blood pressure monitors, glucometers, audiometers, mass spectrometers, and body impedance meters, among others. The mobile communication device serves as a communications link between a portable monitoring device and a remote computing device. In this way, healthcare providers are able to access the patient's real-time health parameters.

U.S. Pat. App. No. 2013/0278552 patent application discloses a detachable apparatus that houses a mobile device, while also providing added interfaces and controls. The apparatus comprises biomedical sensors that acquires the user's physiological parameters. The acquired physiological data can then be transmitted wirelessly to a remote device.

### BRIEF SUMMARY OF THE INVENTION

The present invention relates to a method for using pulse oximetry connected to a portable device case comprising: connecting a portable device to the portable device case via a portable device port, wherein the portable device case comprises a memory and a power supply; connecting a pulse oximeter to the portable device case via a pulse oximeter port; acquiring a pulse oximetry data using the pulse oximeter; storing the acquired pulse oximetry data in a memory in the portable device case; connecting a patient monitoring device to the portable device case via a patient monitoring device port; and transmitting the stored pulse oximetry data to the patient monitoring device.

Thus, the present invention relates to a method for using pulse oximeter connected to a portable device case. The method of the present invention comprises connecting a portable device to the portable device case via a portable device port. To enable patient monitoring, a pulse oximeter is connected to the portable device case via a pulse oximeter port. Then, pulse oximetry data is acquired using the pulse oximeter and is stored in the portable device case's memory. Once the portable device case is connected to a patient monitoring device, the stored pulse oximetry data from the portable device case's memory is transmitted to the patient monitoring device.

The present invention also relates to a system for using pulse oximetry connected to a portable device case comprising: a pulse oximeter; a portable device; and the portable device case comprising: a memory for storing pulse oximetry data, a portable device port for connecting with the portable device, a pulse oximeter port for connecting with the pulse oximeter, and a communications module for transmitting the stored pulse oximetry data to a patient monitoring device.

Thus, the present invention also relates to a system comprising a pulse oximeter, a portable device, and a portable device case. The portable device case further comprises a memory, a portable device port, a pulse oximeter port, and a communications module.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention. In the drawings:

FIG. 1 illustrates a block diagram of a system for processing a patient's pulse oximetry data according to a preferred embodiment of the present invention.

FIG. 2 is a flowchart of the method for processing a patient' pulse oximetry data according to a preferred embodiment of the present invention.

FIG. 3 is a flowchart of the method for a data storage software according to a preferred embodiment of the present invention.

FIG. 4 is a flowchart of the method for a data transfer software according to a preferred embodiment of the present invention.

FIG. 5A illustrates a preferred embodiment of the present invention.

FIG. 5B also illustrates a preferred embodiment of the present invention.

### DETAILED DESCRIPTION OF THE EMBODIMENTS

The following are definitions of terms as used in the various embodiments of the present invention.

The term “portable device” as used herein refers to a hand-held computing device capable of running one or more software. A “portable device,” as used herein, is equipped with communications modules that enables connection to the Internet and other computing devices. Also, a “portable device” is equipped with interfaces that allow attachment of peripheral devices. Examples of “portable devices” are smart phones, PDAs, and tablet computers, among others.

The term “database” as used herein refers to a collection of data and information organized in such a way as to allow the data and information to be stored, retrieved, updated, and manipulated and to allow them to be presented into one or more formats such as in table form or to be grouped into text, numbers, images, and audio data. The term “database” as used herein may also refer to a portion of a larger database, which in this case forms a type of database within a database. “Database” as used herein also refers to conventional databases that may reside locally or that may be accessed from a remote location, e.g., remote network servers. The database typically resides in computer memory that includes various types of volatile and non-volatile computer memory. Memory wherein the database resides may include high-speed random access memory or non-volatile memory such as magnetic disk storage devices, optical storage devices, and flash memory. Memory where the database resides may also comprise one or more software for processing and organizing data received by and stored into the database.

The present invention relates to a method for using pulse oximetry connected to a portable device case comprising: connecting a portable device to the portable device case via a portable device port, wherein the portable device case comprises a memory and a power supply; connecting a pulse oximeter to the portable device case via a pulse oximeter port; acquiring a pulse oximetry data using the pulse oximeter; storing the acquired pulse oximetry data in a memory in the portable device case; connecting a patient monitoring device to the portable device case via a patient monitoring device port; and transmitting the stored pulse oximetry data to the patient monitoring device.

The present invention also relates to a system for using pulse oximetry connected to a portable device case comprising: a pulse oximeter; a portable device; and the portable device case comprising: a memory for storing pulse oximetry data, a portable device port for connecting with the portable device, a pulse oximeter port for connecting with the pulse oximeter, and a communications module for transmitting the stored pulse oximetry data to a patient monitoring device.

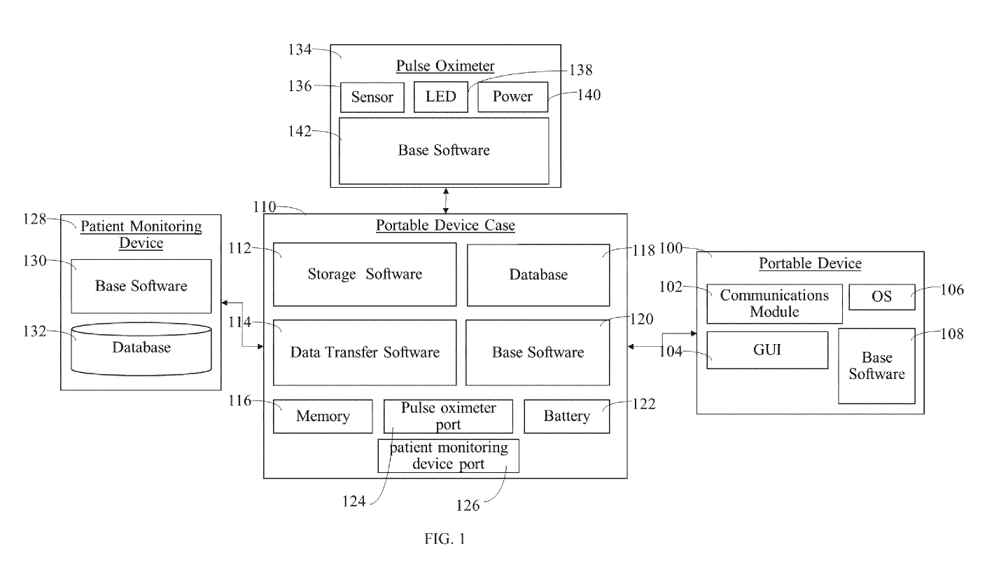

FIG. 1 illustrates a block diagram of the system according to the present invention. A portable device 100 comprises: a communications module 102, a portable device GUI 104, an operating system 106 and a base software 108. The portable device 100 is connected to a portable device case 110. A portable device case 110 is a partial cover encasing the portable device 100 designed such that the built-in controls and inputs (e.g., ports, buttons, screen) of the portable device 100 are not obstructed. The portable device case 110 comprises: a data storage software 112, a data transfer software 114, a base software 120, and a database 118. The portable device case 110 further comprises: a memory 116, a battery 122, a pulse oximeter port 124 and a patient monitoring device port 126. During remote monitoring, a pulse oximeter 134 is connected to the portable device case 110. The pulse oximeter 134 comprises: a sensor 136, an LED array 138, a power module 140, and a base software 142. When the patient visits the hospital, the portable device case 110 is connected to a patient monitoring device 128. The patient monitoring device 128 comprises a base software 130, and a database 132.

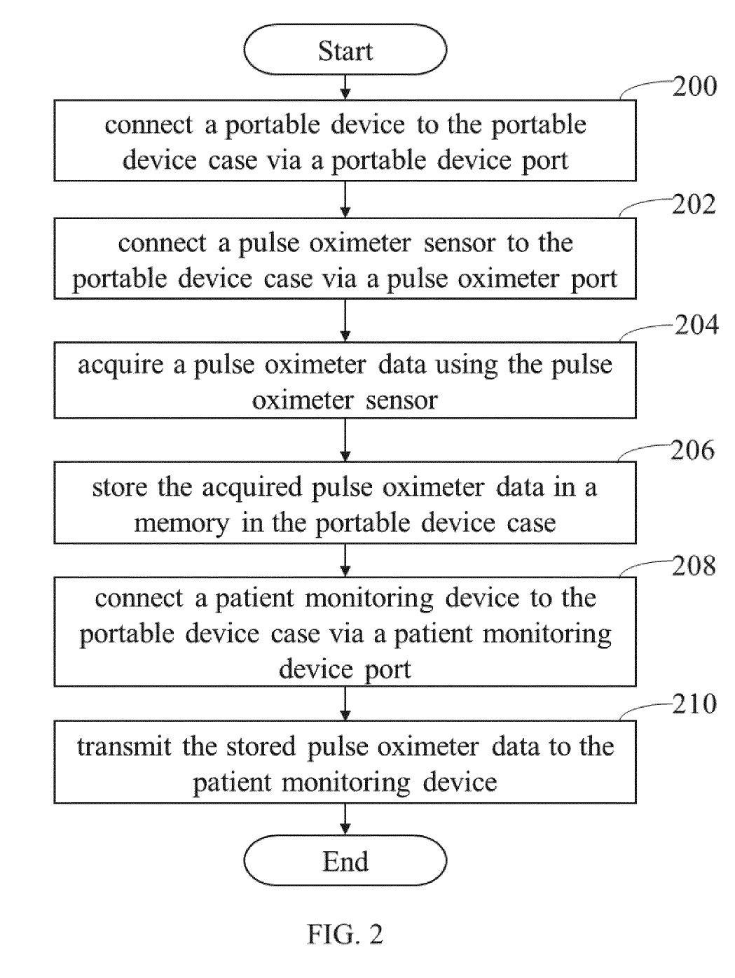

FIG. 2 is a flowchart of a preferred method for using pulse oximetry connected to a portable device. A portable device 100 is first connected to a portable device case 110 (step 200). Preferably, connection between the two components is established via a portable device port. Alternatively, connection from the portable device 100 to the portable device case 110 is done wirelessly via NFC.

As shown in FIG. 2, a pulse oximeter 134 is connected to the portable device case 110 via a pulse oximeter port 124 (step 202). The connection from the pulse oximeter 134 to the portable device case 110 is done via a wired connection. Alternatively, the connection is a wireless connection such as Bluetooth or Wi-Fi. The pulse oximeter 134 is then attached to the patient and pulse oximetry data is acquired (step 204).

The pulse oximeter 134 preferably measures at least one of blood oxygen saturation, pulse rate, perfusion index, and respiration rate. Blood oxygen saturation is a measure of the amount of oxygen carried by hemoglobin in the blood stream. It is usually expressed as a percentage rather than an absolute reading. For example, blood oxygen saturation levels measured immediately after birth can provide a good indicator of a baby's general state of health. Levels below 75% could indicate that the newborn infant may be suffering from some abnormality. To determine a patient's condition, the blood oxygen saturation should be expressed as a percentage of the total hemoglobin that is saturated with oxygen. Under many circumstances, that is the reading that pulse oximeters provides. Acceptable normal ranges for healthy patients range from 95 to 99 percent.

The pulse rate is the number of times the heart beats per minute. The pulse rate is characterized by the peaks in the pleth waveform. Acceptable normal pulse rates for healthy patients range from 60 to 100 beats per minute (bpm). For example, a pulse rate of 40 bpm could indicate a simple low blood pressure or bradycardia. On the other hand, a pulse rate of 100 bpm or more could indicate a fever or low blood sugar. When the pulse rate is irregular, it could indicate arrhythmia which could cause the heart not being able to pump enough blood to the body.

The perfusion index is a ratio of the pulsatile blood flow to the non-pulsatile static blood flow in a patient's peripheral tissue. Perfusion index is an indication of the pulse strength wherein values range from 0.02% for weak pulse strength to 20% for strong pulse strength.

The respiration rate is the number of breaths per minute. The respiration rate is based on changes in the cardiovascular, respiratory and autonomic nervous systems that affects the pleth waveform. These changes can be used to calculate the respiration rate. Acceptable normal ranges for healthy patients range from 12 to 20 breaths/minute. Abnormal ranges of respiration rate may indicate asthma, pneumonia, congenital heart disease or drug overdose.

After acquiring pulse oximetry data (step 204), data are stored in the portable device case's database 118 (step 206). To transmit the pulse oximetry data to the patient monitoring device 128, the portable device case 110 is connected to the patient monitoring device 128 via a patient monitoring device port 126 (step 208). The pulse oximeter port 124 and the patient monitoring device port 126 are preferably standard ports such as microUSB ports. Pulse oximetry data is then transmitted and stored in the patient monitoring device database 132 (step 210).

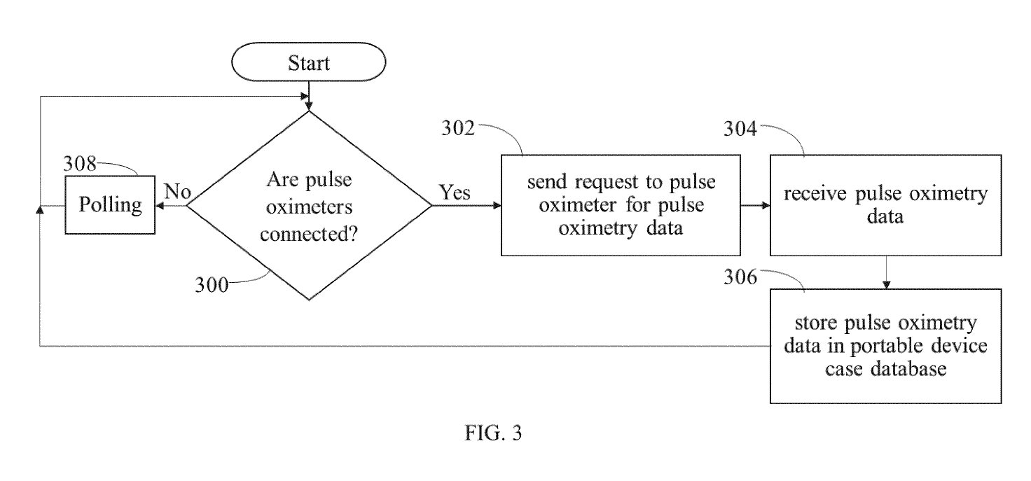

FIG. 3 is flowchart of a method involving a data storage software 112 according to the present invention. First, it is determined if a pulse oximeter 134 is connected to the portable device case 110 (step 300). Provided that there is no pulse oximeter connected, the portable device case 110 continuously polls for a pulse oximeter connection (step 308). Once the portable device case 110 determines a pulse oximeter 134 is connected, a request is sent to the pulse oximeter 134 to start transmitting pulse oximetry data to the portable device case 110 (step 302). The portable device case then starts receiving pulse oximetry data from the pulse oximeter 134 (step 304) and then stores the pulse oximetry data to portable device case database 118 (step 306).

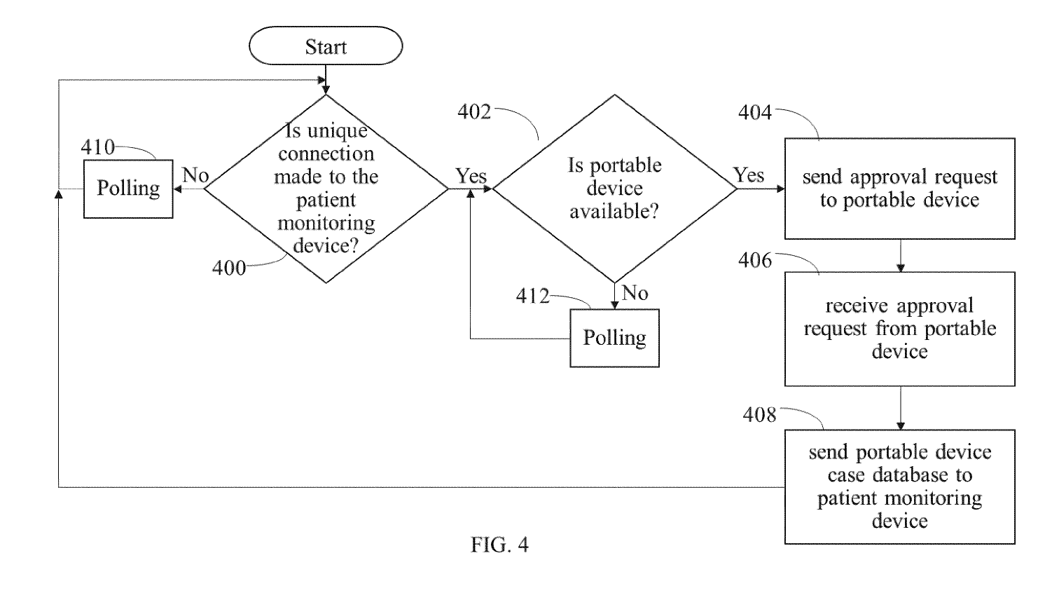

FIG. 4 is a flowchart of a method involving a data transfer software 114 according to the present invention. First, it is determined if a portable device case 134 is connected to a patient monitoring device 128 (step 400). The portable device case 134 continuously polls for a connection to a patient monitoring device 128 provided that there is no patient monitoring device connected (step 410). Once the portable device case 134 determines that it is connected to a patient monitoring device 128, it then determines if the portable device 100 is connected to the portable device case 110 (step 402). The portable device case 110 continuously polls for a connection to a portable device 100 provided that there is no portable device connected yet (step 412). Once the portable device case 110 determines that a portable device 100 is also connected to it, the portable device case 110 sends a request to the portable device 100 (step 404). The request is for the approval of sending the pulse oximetry data stored in the portable device 100 to the patient monitoring device 128. The request is approved through the portable device's graphical user interface 104. Once the approved request is received by the portable device case 110 (step 406), the pulse oximetry data stored in the portable device case's database 118 is transmitted to the patient monitoring device 128 (step 408).

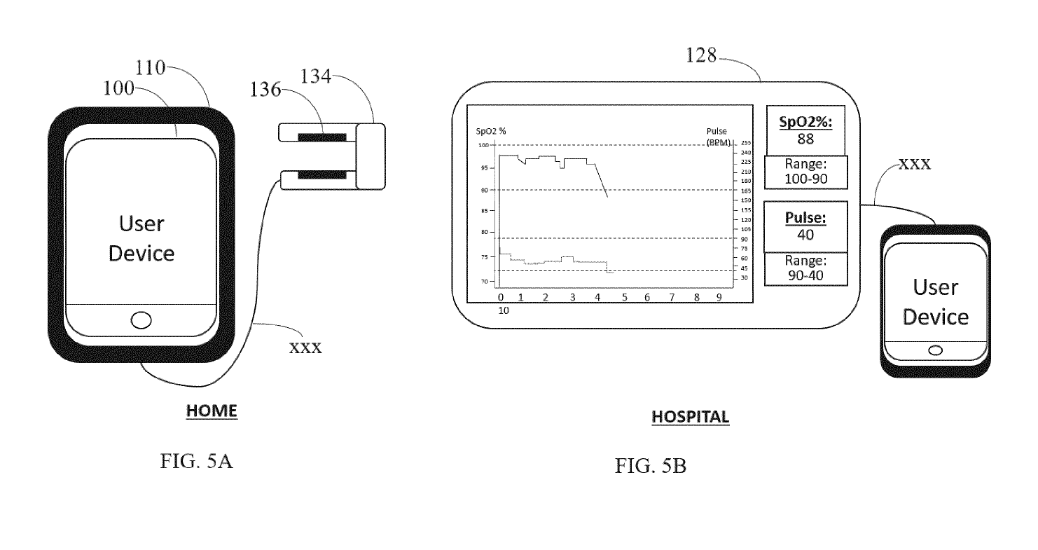

FIG. 5A and FIG. 5B illustrates a preferred embodiment of the system according to the present invention. In FIG. 5A, suppose an outpatient has to have pulse oximetry parameters monitored while he is at home. A pulse oximeter 134 with photosensors and detectors 136 is attached to the outpatient. The pulse oximeter is then connected via a micro-USB cable to a portable device 100 with a portable device case 110. Pulse oximetry data is then gathered and stored into the portable device case 110. When the outpatient visits a physician in the hospital for a check-up, the physician wirelessly connects the outpatient's portable device 100 with a portable device case 110 to a patient monitoring device 128. Pulse oximetry data stored in the portable device case 110 is then transferred to the patient monitoring device 128. The physician can then view the pulse oximetry data on the patient monitoring device 128.

In one embodiment of the present invention, before a patient is discharged from a hospital, a physician sets up the portable device 100 to enable remote monitoring of the patient's pulse oximetry parameters. A range of acceptable pulse oximetry parameter values is set by the physician via the portable device 100 and is transmitted to the portable device case 110. Once the patient gets home, the patient's pulse rate and SpO₂ levels are monitored using the pulse oximeter. During acquisition of pulse oximetry data, the portable device case 110 determines if the patient's acquired pulse oximeter parameter values are within acceptable range. If not, the portable device case 110 sends an alert signal to the portable device 100. The portable device 100, in turn, will trigger an alert to notify the patient that pulse oximetry parameters are lies outside the predefined threshold levels. The alert may be sent in the form of at least one of an audio alert, a visual alert, and a vibrating alert. Optionally, the portable device is prompted to send a message to the physician's portable device. In this way, the physician is able to respond to the alert.

For example, during remote monitoring, the patient experiences a COPD attack. This causes the patient's SpO₂ levels to drop to below normal levels. The portable device case 110 detects the lower than normal SpO₂ level and sends a signal to the portable device 100, which triggers a ringing and vibrating alarm. Simultaneous to the alarm, a text message is sent to the physician's smartphone notifying the physician about the patient's COPD attack.

In addition to detecting higher than normal pulse oximetry parameter values, the portable device case 110 is able to detect if a connected pulse oximeter 134 is malfunctioning. In this case, an alert signal is transmitted to the portable device 100 which, in turn, will trigger an alert to the portable device user.

In another embodiment, the outpatient's pulse oximetry parameters are monitored while the patient performs activities that affect pulse oximetry measurements. In this case, contextual information is added to the pulse oximetry data via user input in a portable device 110. Examples of contextual information are the patient's current physical state, e.g., exercising, eating, and sleeping. Preferably, the contextual information is added by first running a software in the portable device 110 to input contextual information. The inputted contextual information is then transmitted to the portable device case's database 118. Afterwards, the corresponding acquired pulse oximetry data is tagged with the contextual information. For example, a patient's SpO₂ level is monitored while the patient is performing an exercise. Using a smartphone, the patient runs a software to input contextual information by selecting a time option indicating the start and end of an exercise. The contextual information and time data are then transmitted to the portable device case's database 118. Afterwards, the acquired data during the patient's performance of the exercise is tagged with the contextual information.

Environmental conditions can significantly affect pulse oximetry measurements. For example, a smartphone's embedded temperature sensor measures the ambient temperature to be 50° F. (10° C.). At this temperature, the patient may experience local hyperthermia in the fingers, which causes the arteries to constrict. The constriction of arteries lowers the oxygen levels in the blood thus lowering the SpO₂ level. By saving the combined pulse oximetry data with the corresponding temperature data, the effect of ambient temperature can be accounted for.

In a further embodiment, an outpatient is inside a cold room while measuring pulse oximetry parameters. To take into account the effect of environmental factors, data acquired by environmental sensors integrated with the portable device 100 are transmitted to the portable device case's database 118 and are used to further provide context to the acquired pulse oximetry data. Examples of environmental sensors that may be integrated with the portable device 100 are temperature sensors, humidity sensors, and light sensors, among others.

Pulse oximetry measurements are also susceptible to motion which leads to unreliable pulse oximetry data. In one embodiment, the outpatient is jogging while measuring pulse oximetry parameters. To account for motion artifacts, a correction algorithm stored in the portable device case 110 is used to improve pulse oximetry measurement accuracy. In one embodiment, the correction algorithm uses motion sensor data in order to minimize the effect of motion artifacts to pulse oximetry data. Motion sensors can be embedded in at least one of the pulse oximeter 134, portable device case 110, and portable device 100.

The present invention is not intended to be restricted to the several exemplary embodiments of the invention described above. Other variations that may be envisioned by those skilled in the art are intended to fall within the disclosure.

1. A system for using pulse oximetry connected to a portable device case, the system comprising:
a pulse oximeter; and
a portable device; and
a portable device case comprising:
a memory that stores pulse oximetry data, and
a communications module that transmits the stored pulse oximetry data to a patient monitoring device
wherein the portable device case is configured to send an alert signal to the portable device when the acquired pulse oximetry data is not within a predetermined pulse oximetry parameter range.

2. The system of claim 1, wherein the communication module further transmits the stored pulse oximetry data to a cloud database.

3. The system of claim 1, wherein the portable device acquires and transmits contextual data to the portable device case.

4. The system of claim 1, wherein the portable device case further comprises a portable device port that connects to the portable device.

5. The system of claim 1, wherein the portable device case further comprises a pulse oximeter port that connects to the pulse oximeter.

6. A method for using pulse oximetry connected to a portable device case, the method comprising:
receiving pulse oximetry data from the pulse oximeter;
storing the acquired pulse oximetry data in a memory in a portable device case;
connecting a patient monitoring device to the portable device case via a patient monitoring device port;
transmitting the stored pulse oximetry data to the patient monitoring device; and
sending an alert signal to the portable device when the acquired pulse oximetry data is not within a predetermined range.

7. The method of claim 7, further comprising transmitting the stored pulse oximetry data via a communication module of the portable device case to a cloud database.

8. The method of claim 6, further comprising receiving contextual data acquired by the portable device.

9. The method of claim 6, wherein the portable device case is connected to the portable device via a portable device port that connects.

10. The method of claim 6, wherein the portable device case is connected to the pulse oximeter via a pulse oximeter port.
